(19) United States
(12) Patent Application Publication
(10) Pub. No.: US 2025/0283959 A1
(43) Pub. Date: Sep. 11, 2025

(54) METHOD AND APPARATUS FOR MAGNETIC RESONANCE (MR) CONTRAST AGENTS

(71) Applicant: Wayne State University, Detroit, MI (US)

(72) Inventor: Eduard Y. Chekmenev, Troy, MI (US)

(21) Appl. No.: 18/858,953

(22) PCT Filed: Apr. 21, 2023
(86) PCT No.: PCT/US2023/019375

§ 371 Date: Oct. 22, 2024

Related U.S. Application Data
(60) Provisional application No. 63/334,118, filed on Apr. 23, 2022.

(51) Int. Cl. G01R 33/28; A61K 49/18

(52) U.S. Cl. CPC G01R 33/282; A61K 49/1896

(57) ABSTRACT

A magnetic resonance (MR) contrast agent system configured to be usable within a magnetic resonance imaging (MRI) system as the MRI system is scanning. The MR contrast agent system includes a non-magnetic container having a propylene-parahydrogen gas mixture therein, a non-magnetic gas valve, a non-magnetic reactor, and a non-magnetic mouthpiece. The non-magnetic container is coupled to the non-magnetic gas valve and the non-magnetic gas valve is coupled to the non-magnetic reactor. The non-magnetic reactor is configured to convert the propylene-parahydrogen gas mixture to a hyperpolarized gas as the propylene-parahydrogen gas mixture passes through the non-magnetic reactor. The non-magnetic mouthpiece is configured to allow passage of the hyperpolarized gas into a subject.

### CROSS-REFERENCE TO RELATED APPLICATIONS

This application claims priority to U.S. Provisional Application No. 63/334,118 filed on Apr. 23, 2022, the contents of which is hereby incorporated by reference in its entirety.

### GOVERNMENT LICENSE RIGHTS

The invention was made with government support under the following: Contract No. W81XWH-20-10576 awarded by Department of Defense, CDMRP.

### FIELD OF TECHNOLOGY

An improved contrast agent system and method are disclosed. Improvements are applicable to the field of magnetic resonance (MR) technology.

### BACKGROUND

Biomedical imaging is an important tool in the study of anatomy and physiology of humans. For example, biomedical imaging is employed for disease diagnostics, monitoring of the disease progression, and assessing response to treatment among other things. Magnetic resonance imaging (MRI), for example, is widely used due to its non-invasive nature for obtaining structural, functional, and dynamic information from subject tissues of interest. Nuclear magnetic resonance (NMR) is regarded as a low sensitive spectroscopic technique due to its low degree of nuclear spin alignment or spin polarization. For example, protons are generally polarized to only 1×10⁻⁵ at 3 tesla (T) at room temperature. To remedy MRI sensitivity limitations due to low nuclear spin polarization, conventional MRI is generally performed with highly abundant water protons. However, a substantial polarization enhancement by 4-6 orders of magnitude can be transiently created via NMR hyperpolarization techniques. As a result of massive nuclear spin polarization boost that result from such techniques, it becomes possible to sense low-concentration hyperpolarized (HP) exogenous contrast agents such as HP gases. As such, inhalable HP gases may be used for imaging things such as void spaces (e.g., pulmonary imaging).

Hyperpolarization allows rapid image acquisition because imaging sequences obviate spin-recovery that is often required in conventional MRI. HP noble gas (including 129Xe) may be prepared in large enough quantities for clinical studies. Further, HP noble gases such as 129Xe gas can be inhaled as a contrast agent, and a functional pulmonary 3D image can be recorded during a single patient breath-hold (e.g., a 3-10 second breath-hold).

While 129 Xe contrast agents are beneficial, preparation of hyperpolarized 129Xe contrast agents often require the presence of complex on-site infrastructure. Once hyperpolarizing devices dispense an HP contrast agent, the HP state decays to thermal equilibrium. Due to the non-replenishable characteristic of the HP state, and because the lifetime of HP noble gases is limited, the long-range transportation of these HP contrast agents to the administration site remains challenging. As a result, production of HP noble gases is often established on site, which creates other challenges, such as an on-site requirement of expertise in production of HP media as well as the cost associated with production of such media.

A common inhalable HP contrast is 129Xe, which is generally hyperpolarized on site via Spin Exchanged Optical Pumping (SEOP). HP 129Xe is extensively studied for a wide range of pulmonary imaging applications. Despite the success of HP 129Xe in the research setting to diagnose a wide range of lung conditions, the clinical translation of this HP contrast agent and its production technology faces translation barriers. In addition to the instrumentation complexity discussed above, the SEOP hyperpolarizers are considered expensive (e.g., $0.3M-0.6M). Further, conventional MRI scanners can often only image protons and are generally not suited for HP 129Xe imaging without complex and costly upgrades. As such, higher-end or modified MRI scanners often need to be employed.

Accordingly, there is a need for a system and method for the creation of HP gaseous contrast agents that can be used in the conventional MRI setting.

### BRIEF DESCRIPTION OF THE DRAWINGS

FIG. 1 illustrates an exemplary magnetic resonance (MR) contrast agent system;

FIG. 2 illustrates another exemplary MR contrast agent system;

FIG. 3 illustrate an exemplary technique for manufacturing an MR contrast agent system; and

FIG. 4 illustrates an exemplary technique for administering a magnetic resonance imaging (MRI) contrast agent.

### DETAILED DESCRIPTION

Parahydrogen-Induced Polarization (PHIP) is employed to quickly create a batch of proton magnetization that can be inhaled or injected into a living subject (including human) to serve as a contrast agent during Magnetic Resonance Imaging (MRI) scanning. Further, such implementations may also be employed on objects (e.g., for scanning non-magnetic packages or devices). One exemplary technique for creating such a contrast agent includes pairwise parahydrogen addition to propylene gas to create hyperpolarized propane gas, which can be used as an inhalable or injectable contrast agent for pulmonary and other MRI applications. The term “hyperpolarized (HP) propane” refers to propane with increased nuclear spin polarization of its proton nuclear spins over natural or equilibrium levels.

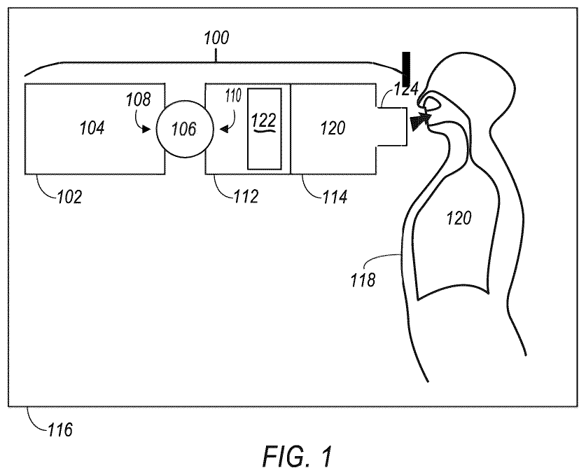

FIG. 1 is a block diagram of an exemplary HP contrast agent system 100 in use. The exemplary system includes the following: a non-magnetic container 102 having a propylene-parahydrogen gas mixture 104 therein; a non-magnetic gas valve 106 having a first end 108 and a second end 110, where the first end 108 is coupled to the non-magnetic container 102; a non-magnetic reactor 112 coupled to the second end 110 of the non-magnetic valve 106, and a gas passage or exit (e.g., a non-magnetic mouthpiece 114) coupled to the non-magnetic reactor 112.

The gas mixture 104 may be a compressed propylene-parahydrogen gas mixture that is loaded into the container 102 (e.g., a high-pressure non-magnetic container). The gas flow valve 106 may be actuated to load the propylene-parahydrogen mixture 104 into the non-magnetic container 102 prior to coupling the gas valve 106 to the reactor 112. The loading of the container 102 may be carried out at a remote facility.

Following the loading of the compressed propylene-parahydrogen mixture 104 into the container 102, the valve 106 may be self-sealed. The properties of both the non-magnetic container 102 and the compressed propylene-parahydrogen mixture 104 help to preserve the potency of the loaded mixture 104 for a sufficiently long period of time. For example, if paramagnetic impurities in the container 102 are avoided, back-conversion of parahydrogen to normal hydrogen can be minimized, thus maximizing hyperpolarization efficiency later. As such, by employing non-magnetic container substantially free of paramagnetic impurities, the shelf-life of the propylene-parahydrogen may be extended to, for example, at least 12.8 days (±2.6 days). Similarly, avoiding paramagnetic impurities such as air or molecular oxygen in the gas mixture 104 may also minimize back-conversion of parahydrogen to normal hydrogen, thus maximizing the lifetime of hyperpolarization potency even further.

Further, different shapes and sizes of the non-magnetic container 102 can be employed to minimize the back-conversion of parahydrogen to normal hydrogen. For example, canister shapes and/or sizes that increase or maximize the mean free path of gas flow of the gas mixture 104 in the container 102 can minimize the back-conversion of parahydrogen to normal hydrogen. For example, in some contexts a spherical container may maximize the mean free path of gas flow of the gas mixture 104.

With continued reference to FIG. 1, due to the non-magnetic properties of the HP contrast agent system 100, the system 100 can be placed inside, or in the proximity, of an MRI scanner 116 (e.g., a conventional MRI scanner) while scanning occurs. While a subject 118 is in the MRI scanner 116, the mouthpiece 114 may be placed in the mouth of the subject 118. A simple breath-hold may actuate the valve 106, thus causing a stream of the gas mixture 104 to flow through the reactor 112, thus causing hyperpolarized propone gas 120 to exit and enter the subject 118 via the mouthpiece 114. Other implementations may employ a valve (e.g., non-magnetic gas valve 106 or another valve) that may be physically actuated by a technician or the subject to cause the hyperpolarized propone gas 120 to pass through the reactor 112 and enter the subject 118.

Regardless of the manner in which the gas mixture 104 is caused to leave the canister 102, the reactor 112 causes pairwise parahydrogen addition in the gas mixture 104 as it flows through the reactor 112 so that the subject 118 receives a hyperpolarized propane contrast agent 120. The reactor 112 (e.g., a flow reactor) includes a parahydrogen-Induced Polarization (PHIP) catalyst 122, which renders some degree of pairwise addition to the parahydrogen and propylene gas mixture 104 as it flows through or over the catalyst 122. For example, the reactor 112 may include a 1% Rh/TiO2 catalyst. Other catalysts, however, may be employed.

The mouthpiece 114 may have an overpressure protection component 124 coupled thereto or integrated therein. Further, the gas flow valve 106 may have a flow rate from, for example, 10 standard cubic centimeters per second (sccs) to 10,000 sccs.

Once the hyperpolarized gas 120 enters the subject 118, the gas serves as a contrast agent in the subject 118 during MRI scanning. For example, the HP propane gas can be used to produce, for example, slice-selective MRI images using a conventional MRI scanner (e.g., a 0.35 T MRI scanner) without modifications. The MRI scan can be repeated several times until the hyperpolarized state of the hyperpolarized propane gas decays back to the equilibrium state.

A wide range of the ratios (e.g., 1:1 to 2:1) of parahydrogen to propylene in the compressed parahydrogen-propylene gas mixture 104 may be employed. The total pressure of the compressed parahydrogen-propylene gas mixture 104 may be, for example, 100 bar or less. Further, the gas mixture 104 may be stored for at least several days without a significant loss of the parahydrogen fraction. In some examples, the gas mixture 104 may be stored up to 72 days without a significant loss of the parahydrogen fraction, thus still being usable as an effective HP contrast agent. In other examples, the gas mixture 104 may be stored up to 365 days and still be usable in instances that can capitalize on the reduced parahydrogen fraction.

Following the production of HP propane gas 120 and release of residual gas mixture 104, the system 100 may be discarded (i.e., a one-time or disposable utility) or re-used. Alternatively, one or more components (e.g., components 102-124) may be discarded and/or re-used. For example, the mouthpiece 114 can be discarded, while high-pressure non-magnetic container 102 may be recharged with a new batch of compressed gas mixture 104 and reused together with valve 106 and reactor 112. As another example, the gas mixture 104, the valve 106, and the mouthpiece 114 may be discarded and the reactor 112 with its catalyst 122 may be re-used.

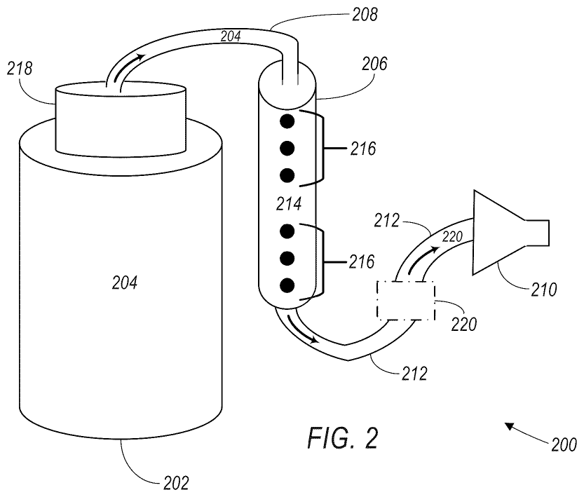

With reference now to FIG. 2, another exemplary contrast agent system 200 is illustrated. The system 200, which may be handheld, is non-magnetic and includes a nonmagnetic container 202 (e.g., a disposable high-pressure nonmagnetic container) having a parahydrogen-propylene gas mixture 204 therein. The container 202 is coupled to a nonmagnetic reactor 206 via a first non-magnetic tubing 208 and the non-magnetic reactor 206 is coupled to a nonmagnetic mouthpiece 210 (or other exit for gas) via a second non-magnetic tubing 212.

The reactor 206 may include a food-grade copper tubing (non-magnetic) filled with, for example, a parahydrogen-Induced Polarization (PHIP) catalyst 214 and non-magnetic beads 216 or the like. The catalyst 214 may, for example, include 100 mg of 1% Rh/TiO2. Since the reaction of parahydrogen addition to propylene is exothermic, the beads 216 (e.g., copper beads) may be employed to improve thermal management of the reactor. The beads 216 of FIG. 2 are merely exemplary and more or less beads than those shown may be employed. Further, other types of non-magnetic thermal management components may instead be employed.

Once a non-magnetic valve 218 coupled to the container 202 is actuated, the parahydrogen-propylene gas mixture 204 passes through the first tubing 208 to the reactor 206, where the catalyst 214 causes the parahydrogen-propylene gas mixture 204 passing thereover to convert to an HP propane gas. As such an HP propane gas 220 fills the second tubing 212 as it passes to the mouthpiece 210 and then into an object or a subject (e.g., the subject 118 of FIG. 1) and MR imaging occurs to capitalize on the HP propane gas that is now within the object or subject.

It is noted that once the gas mixture 204 in the canister 202 is hyperpolarized via the reactor 206, the HP gas 220 may remain in a usable for MRI applications for a period of time (e.g., 0.01-10 minutes). Accordingly, after the first HP propane gas dose inhaled by the subject is exhaled, any remaining HP gas 220 in the second tubing 212 may still serve as an effective contrast agent. As such, a second dose of the HP gas, even if mixed with HP gas 220 that may remain in the second tubing 212 from the previous dose, still serves as an effective contrast agent.

Due to the non-magnetic nature of the contrast agent system 200, the system may be placed in, or in the proximity of, an MRI scanner (e.g., the MRI scanner 116 of FIG. 1). Further, due to the extent that the system 200 is able to hyperpolarize the nuclear spins of the gas mixture 204 (e.g., hyperpolarization of nuclear spins to 0.01% or more), a conventional MRI scanner (e.g., a 3.0 T MRI scanner) may be employed without any modifications to the hardware or the software of the MRI scanner. A variety of MRI scanner pulse sequences may be employed for fast imaging before the subject expels (e.g., breath-holds of 3-10 seconds) the HP gas or the contrast agent loses HP potency (i.e., HP decay to equilibrium). For example, multiple 2D-slice-selective MRI scans with sub-second and sub-millimeter temporal and spatial resolution that employ gradient-echo (GRE) pulse sequences may be used. As another example, 2D multi-slice MRI scans with sub-second and sub-millimeter temporal and spatial resolution, respectively, using eco-planar imaging (EPI) pulse sequence may be employed. Of course, other MRI techniques may also be employed to take advantage of the breath-hold time range.

While only one valve 218 was discussed above with respect to FIG. 2, other examples may include one or more additional valves. For example, FIG. 2 illustrates a second non-metallic valve 220 between the reactor 206 and the mouthpiece 210 (or gas exit). The second valve 220 could be controlled by a technician or a subject.

Further, while the exemplary system 200 of FIG. 2 includes tubing 208, 212, other exemplary systems may have different configurations. For example, the tubing 208 between the canister 202 and the reactor 206 may be avoided. That is, the canister 202 may be coupled directly to the reactor 206. Additionally, or alternatively, the tubing 212 between the reactor 206 and the mouthpiece 210 may be avoided. That is, the reactor 206 may instead be coupled directly to the mouthpiece 210 or some other component that allows the HP gas 220 to leave the system.

The systems described above (e.g., the system 100 of FIG. 1 and the system 200 of FIG. 2) may be used, for example, for inhalation of hyperpolarized propane gas by a subject to perform MRI (e.g., pulmonary function MRIs). Images such as pulmonary function images (obtained using hyperpolarized propane contrast agent gas) may, for example, be co-registered with the anatomical images obtained on the exemplary MRI device during the same imaging session, or from a different scanner taken during a different session. The imaging session may, for example, last less than 1 minute. The technique can yield minimal patient discomfort while providing a non-invasive, high-resolution MRI scan of lung function using no ionizing radiation.

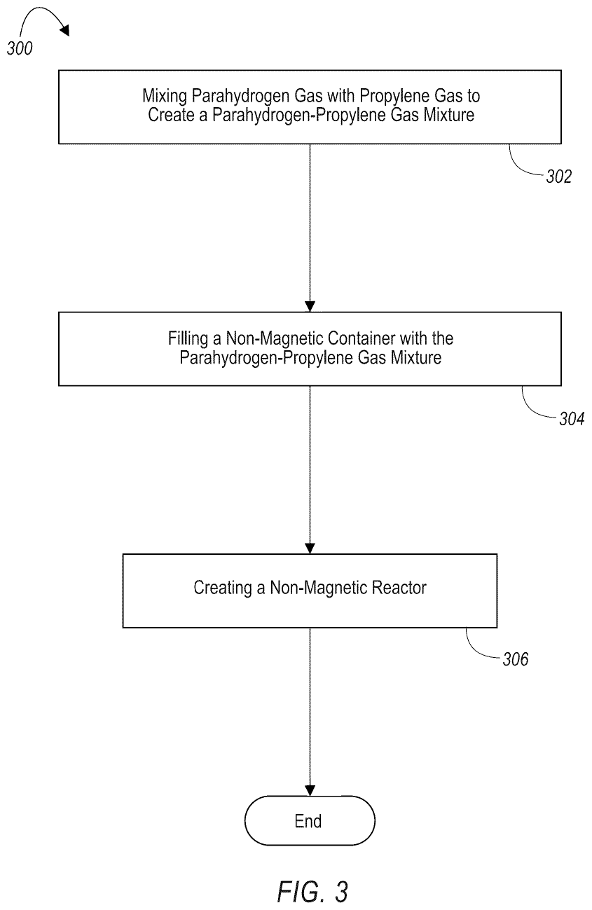

Referring now to FIG. 3, an exemplary technique 300 for manufacturing a magnetic resonance (MR) contrast agent system is shown. The exemplary technique 300 begins at block 302 where mixing parahydrogen gas with propylene gas to create a parahydrogen-propylene gas mixture is carried out. To enhance the shelf-life of the parahydrogen-propylene gas mixture, the non-magnetic container may be free of paramagnetic impurities. Likewise, the parahydrogen-propylene gas mixture may also be free of paramagnetic impurities. As such, the parahydrogen-propylene gas mixture in the non-magnetic container may have a usable shelf-life of 365 days or less.

After the gas is mixed, process control then proceeds to block 304, where filling a non-magnetic container with the parahydrogen-propylene gas mixture occurs. Filling the non-magnetic container with the parahydrogen-propylene gas mixture may include pressurizing the parahydrogen-propylene gas mixture. For example, the parahydrogen-propylene gas mixture may be pressurized within the non-magnetic container to, for example, 100 bar or less.

With regard to the non-magnetic container, it may have a valve coupled thereto. The valve may be configured to allow gas flow in a range of, for example, 10-10,000 standard cubic centimeters per second.

With continued reference to FIG. 3, at block 306, creating a non-magnetic reactor is carried out. The non-magnetic reactor is configured to convert the parahydrogen-propylene gas mixture to a hyperpolarized gas as the parahydrogen-propylene gas mixture passes through the non-magnetic reactor, where the hyperpolarized gas is an MRI contrast agent that enhances images from an MRI scan. It is noted that, in other examples, the non-magnetic reactor may be created 306 before the canister is filled 304 or before the gases are mixed 302.

The non-magnetic reactor includes a catalyst to convert the gas mixture passing therethrough into an HP gas that is effective as a contrast agent. A variety of catalysts (e.g., a Rh/TiO2 catalyst) and catalyst structures may be employed. For example, the catalyst may include rhodium (Rh) nanoparticles on titanium oxide (IV) support, thus enabling the conversion of the gas mixture to an HP gas. As another example, the catalyst may include rhodium (Rh) nanoparticles on aluminum oxide support, thus enabling the conversion of the gas mixture to an HP gas. Other catalysts and catalyst structures that cause the gas mixture to convert to an HP gas (e.g., a HP propane gas) may also be employed.

After the reactor is created, process control proceeds to an end. Other techniques, however, may continue before coming to an end. For example, a mouthpiece may be coupled to the reactor. Alternatively, as another example, another component different than a mouthpiece may be coupled to the reactor. Such a component may, for example, allow passage of the HP gas into an object or a different part of a subject's body.

The filled container, reactor, mouthpiece, and/or any tubing or couplings employed may be shipped to a facility carry out the MRI scan. Further, the components may be individually shipped. For example, since the reactor may be employed for several uses, the facility may need to replenish stock of filled containers more often. As such, orders may be made and filled just for one or more filled containers. Of course the other components (e.g., the reactor) may be shipped individually or as a group as well.

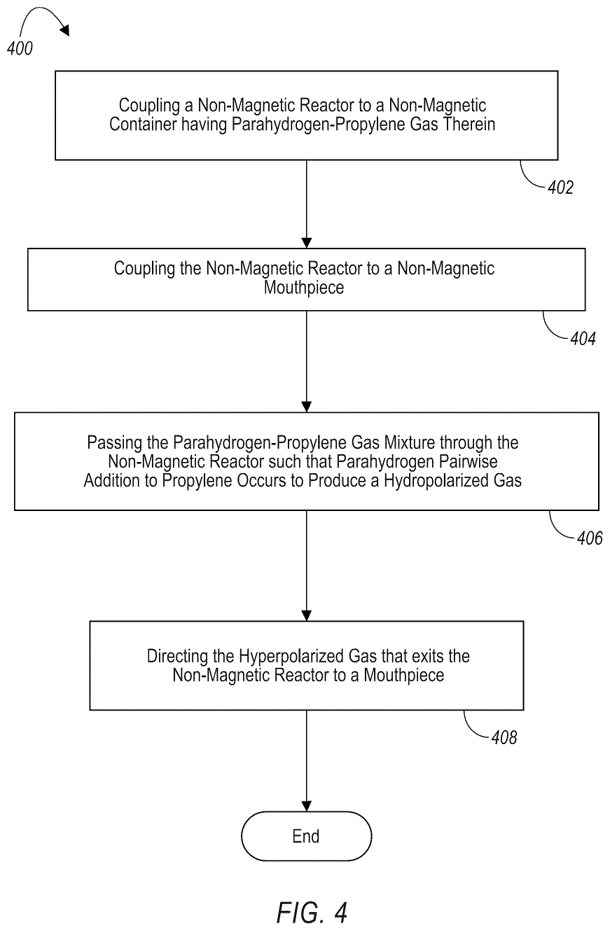

With reference now to FIG. 4, an exemplary technique 400 for administering a magnetic resonance imaging (MRI) contrast agent is shown. Starting at block 402, the exemplary technique 400 includes coupling a non-magnetic reactor to a non-magnetic container, where the non-magnetic container has a parahydrogen-propylene gas mixture therein. The container may be coupled directly to the reactor or the container may be coupled to the reactor via other components (e.g., via tubing). Process control then proceeds to block 404, where coupling the non-magnetic reactor to a non-magnetic mouthpiece (or another component that allows the HP gas to exit the system) occurs. Similarly, the reactor may be coupled directly to the mouthpiece or other gas passage, or the reactor may be coupled to the mouthpiece or other gas passage via other components (e.g., via tubing).

While the technique 400 of FIG. 4 shows that the reactor is coupled to the container before it is coupled to the mouthpiece, other techniques may change the order. Indeed, the mouthpiece may instead be coupled to the reactor before, or at the same time, the reactor is coupled to the canister/container.

Nonetheless, while a subject is in an MRI scanner, the mouthpiece can be placed in a subject's mouth by a technician, or the subject may place the mouthpiece in their own mouth. Alternatively, if an object different than a human is to be scanned, open-ended tubing or the like may be used to allow the system to pass HP gas to the object.

At block 406, passing the parahydrogen-propylene gas mixture through the non-magnetic reactor such that parahydrogen pairwise addition to propylene occurs to produce a hyperpolarized gas (e.g., HP propane gas) is carried out. That is, the gas mixture is passed through the reactor so that it is converted to an HP gas (e.g., HP propane gas). The HP gas may have nuclear spins hyperpolarized to, for example, 0.01% or more. The non-magnetic reactor may have a parahydrogen-Induced Polarization (PHIP) non-magnetic catalyst therein to cause hyperpolarization of the gas mixture. The catalyst may, for example, include Rh/TiO2.

Passing the parahydrogen-propylene gas mixture through the reactor may be carried out in a variety ways. For example, a breath-hold by the subject may actuate a valve on the canister or somewhere else in the system to cause the gas mixture to pass through the reactor. Alternatively, a valve may be actuated by a technician or patient/subject to cause the gas mixture to pass through the reactor and become hyperpolarized.

As the gas mixture passes through the reactor and becomes hyperpolarized, directing the hyperpolarized gas that exits the non-magnetic reactor to a mouthpiece (or other opening) coupled to a subject or object inside an MRI scanner (e.g., a 1 milli-Tesla to 10 Tesla MR scanner) occurs at block 408 as MRI scanning begins or continues. As such, at least some of the MR scanning is carried out while the hyperpolarized gas is within the subject or object. Directing the HP gas from the reactor through the mouthpiece and into the container can occur in a variety of ways. For example, non-magnetic flexible tubing may be employed to direct the HP gas from the reactor to the mouthpiece. Alternatively, the mouthpiece may be directly connected to the reactor or integrated inside the reactor.

Due to the non-magnetic nature of the MR contrast agent system (e.g., the canister, reactor, any tubing present, and mouthpiece), the system may be placed within or near any MR scanner without the risk of it being determinately affected by the magnetic fields of the MR scanner as it operates.

After the HP gas is directed to the mouthpiece or some other type of exit for the HP gas, the technique 400 comes to and end. Other techniques, however, may continue.

An exemplary advantage of the systems and methods described herein is that they enable an ultra-low-cost disposable handheld system for parahydrogen transportation and utilization in proximity to the MRI scanner. Each component of the system may be non-magnetic to enable operation of the system for parahydrogen-based hyperpolarization near or inside an MRI scanner. In addition, due to the effectiveness of the system, conventional MRI scanners (i.e., MRI scanners equipped with proton-only detection capabilities) may be employed without the need for hardware and/or software modifications.

Further, the system's gas container may be loaded with one or more doses of the parahydrogen-propylene gas mixture for production of hyperpolarized contrast agents. The potency of compressed parahydrogen-propylene gas mixture may be retained for a sufficiently long period of time to allow for at least short-term temporary storage and transportation of the system from the manufacturing/dispensing site to the utilization site (MRI imaging facility). Accordingly, the MRI scanning facility need not produce their own gas mixtures (pre-cursor).

Yet another exemplary advantage of the system is that it may be handheld and/or disposable. Accordingly, robust pre-clinical and clinical utilizations are enabled.

While the preceding discussion is generally provided in the context of medical imaging, it should be appreciated that the present techniques are not limited to such medical contexts. The provision of examples and explanations in such a medical context is to facilitate explanation by providing examples of implementations and applications. The disclosed approaches may also be utilized in other contexts, such as the non-destructive inspection of manufactured parts or goods (i.e., quality control or quality review applications), and/or the non-invasive inspection or imaging techniques. As such, instead of employing a mouthpiece, other components may instead be employed to facilitate to passage of HP gas.

With further regard to FIGS. 1-4 and the examples, processes, systems, methods, techniques, heuristics, and etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain examples, and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary. Further, the use of terms such as “first,” “second,” “third,” and the like that immediately precede an element(s) do not necessarily indicate sequence unless set forth otherwise, either explicitly or inferred through context.

While the disclosed materials have been described in detail in connection with only a limited number of embodiments, it should be readily understood that the embodiments are not limited to such disclosed embodiments. Rather, that disclosed can be modified to incorporate any number of variations, alterations, substitutions or equivalent arrangements not heretofore described, but which are commensurate with the spirit and scope of the disclosed materials. Additionally, while various embodiments have been described, it is to be understood that disclosed aspects may include only some of the described embodiments. As such, that disclosed is not to be seen as limited by the foregoing description, but is only limited by the scope of the appended claims.

1. A magnetic resonance (MR) contrast agent system comprising:
a non-magnetic container having a propylene-parahydrogen gas mixture therein;
a non-magnetic gas valve coupled to the non-magnetic container;
a non-magnetic reactor coupled to the non-magnetic gas valve, wherein the non-magnetic reactor is configured to convert the propylene-parahydrogen gas mixture to a hyperpolarized gas as the propylene-parahydrogen gas mixture passes through the non-magnetic reactor; and
a non-magnetic mouthpiece coupled to the non-magnetic reactor, wherein the non-magnetic mouthpiece is configured to allow passage of the hyperpolarized gas into a subject, and wherein the MR contrast agent system is configured to be usable within a magnetic resonance imaging (MRI) system as the MRI system is scanning.

2. The MR contrast agent system of claim 1 further comprising a second non-magnetic gas flow valve between the non-magnetic mouthpiece and the non-magnetic reactor.

3. The MR contrast agent system of claim 1, wherein the non-magnetic reactor comprises a parahydrogen-Induced Polarization (PHIP) catalyst.

4. The MR contrast agent system of claim 3, wherein the PHIP catalyst comprises Rh/TiO2, and wherein the hyperpolarized gas is a hyperpolarized propane gas.

5. The MR contrast agent system of claim 3, wherein the non-magnetic reactor further comprises non-magnetic metallic beads configured to dissipate heat as the propylene-parahydrogen gas mixture reacts with the PHIP catalyst.

6. The MR contrast agent system of claim 3, wherein non-metallic reactor comprises copper.

7. The MR contrast agent system of claim 1, wherein the non-magnetic container is free of paramagnetic impurities.

8. The MR contrast agent system of claim 1, wherein the propylene-parahydrogen gas is free of paramagnetic impurities.

9. A method of administering a magnetic resonance imaging (MRI) contrast agent comprising:
coupling a non-magnetic reactor to a non-magnetic container, wherein the non-magnetic container has a parahydrogen-propylene gas mixture therein;
coupling the non-magnetic reactor to a non-magnetic mouthpiece;
passing the parahydrogen-propylene gas mixture through the non-magnetic reactor such that parahydrogen pairwise addition to propylene occurs to produce a hyperpolarized gas; and
directing the hyperpolarized gas that exits the non-magnetic reactor to a mouthpiece coupled to an object that is inside an MRI scanner such that MRI scanning occurs while the hyperpolarized gas is within the object.

10. The method of claim 9, wherein the hyperpolarized gas is a hyperpolarized propane gas that includes nuclear spins hyperpolarized to 0.01% or more, and wherein the non-magnetic reactor comprises a parahydrogen-Induced Polarization (PHIP) catalyst therein, and wherein the object is a subject.

11. The method of claim 10 further comprising placing the non-magnetic reactor and the non-magnetic container in the MRI scanner such that the MRI scanning occurs while the non-magnetic reactor and the non-magnetic container are in the MRI scanner, and wherein the PHIP catalyst comprises Rh/TiO2.

12. The method of claim 10, wherein at least the non-magnetic container is substantially free of paramagnetic impurities, and wherein a magnetic field of the MRI scanner is in a range from 1 milli-Tesla to 10 Tesla.

13. The method of claim 10, wherein the parahydrogen-propylene gas mixture in the non-magnetic container is substantially free of air and molecular oxygen.

14. The method of claim 10, wherein the MRI scanner has proton-only detection capabilities.

15. A method of manufacturing a magnetic resonance imaging (MRI) contrast agent system comprising:
mixing parahydrogen gas with propylene gas to create a parahydrogen-propylene gas mixture;
filling a non-magnetic container with the parahydrogen-propylene gas mixture; and
creating a non-magnetic reactor to convert the parahydrogen-propylene gas mixture to a hyperpolarized gas as the parahydrogen-propylene gas mixture passes through the non-magnetic reactor, wherein the hyperpolarized gas is an MRI contrast agent that enhance images from an MRI scan.

16. The method of claim 15, wherein the non-magnetic container is free of paramagnetic impurities.

17. The method of claim 16, wherein filling the non-magnetic container includes pressurizing the parahydrogen-propylene gas mixture inside the non-magnetic container to 50 bar or less, and wherein the parahydrogen-propylene gas mixture is free of paramagnetic impurities.

18. The method of claim 15, wherein the non-magnetic container has a valve coupled thereto that is configured to allow gas flow in a range of 10-10,000 standard cubic centimeters per second, and wherein the parahydrogen-propylene gas mixture in the non-magnetic container has a usable shelf-life of 365 days or less.

19. The method of claim 15, wherein the non-magnetic reactor comprises rhodium (Rh) nanoparticles on titanium oxide (IV) support.

20. The method of claim 15, wherein the non-magnetic reactor comprises rhodium (Rh) nanoparticles on aluminum oxide support.
